(19) United States
(12) Patent Application Publication
(10) Pub. No.: US 2007/0254858 A1
(43) Pub. Date: Nov. 1, 2007

(54) Contraceptive and Acne Medication Combination and Treatment of Acne and Other Diseases with Reduced Side Effects

(76) Inventor: Peter J. Cronk, Moorestown, NJ (US)

Correspondence Address: DUANE MORRIS, LLP;IP DEPARTMENT, 30 SOUTH 17TH STREET, PHILADELPHIA, PA, 19103-4196, US

(21) Appl. No.: 11/462,762

(22) Filed: Aug. 7, 2006

Related U.S. Application Data
(63) Continuation-in-part of application No. 11/380,446, filed on Apr. 27, 2006.

(51) Int. Cl. A61K 31/56; A61K 31/57; A61K 31/203

(52) U.S. Cl. 514/170; 514/559

(57) ABSTRACT

The present invention provides pharmaceutical compositions for the treatment of acne, psychotic illnesses, such as schizophrenia, cancer, such as cancer of the head, neck and lung, and emphysema, comprising co-administering a therapeutically effective amount of isotretinoin and a contraceptive in a contraceptively effective dosage. The contraceptive preferably does not contain 100 wt % progestogen. Methods of treating acne and unit dosage delivery systems are also provided. Also provided is an improved method of treating acne, and other diseases, comprising providing a blister pack comprising at least about 28 separate daily dosage units of a therapeutically effective amount of isotretinoin, or other retinoic acid derivative equivalent, and at least about 21-28 additional daily dosage units of an oral contraceptive in a contraceptively effective amount, selected from the group consisting essentially of: estrogen, estrogen and progestogen, mifepristone, or a combination thereof, whereby for at least about 21 successive days, said oral dosage unit of isotretinoin, or other retinoic acid derivative equivalent, and said oral dosage unit of said oral contraceptive are administered to a female of child-bearing age. Patch, inhaler, nebulizer, and injection formulations for the preferred active ingredients are also provided.

### CROSS-REFERENCE TO RELATED APPLICATIONS

The present application is a continuation-in-part of U.S. application Ser. No. 11/380,446, filed Apr. 27, 2006 (Attorney Docket D0968-00072).

### FIELD OF THE INVENTION

The present invention is related to acne treatments and compositions for treating acne, and especially those that are designed to minimize birth defects in patients being treated with isotretinoin.

### BACKGROUND OF THE INVENTION

The treatment of acne can be summarized into established tiers of care, beginning with topical remedies including topical benzoyl peroxide 5-10% and/or topical antibiotics and retinoic acid (Retin-A®), followed by tretinoin, oral antibiotics, hormone-related therapy, and finally, isotretinoin (Accutane®, Amnesteem®, Claravis® and Sotret®).

Oral antibiotics, such as tetracycline, erythromycin, taken by mouth, are usually a critical step in the treatment of acne. Antibiotics limit the growth of acne and decrease redness and inflammation. Over time, however, resistance to these drugs can occur. Tetracyclines, including doxycycline and minocycline are the most common antibiotics used in the treatment of acne. Usually these are prescribed once or twice daily and are taken on an empty stomach for improved absorption. These medications often increase one's sensitivity to sun's ultraviolet rays, and rash is a common side effect in those exposed to sunlight while taking the medications. They are obtained only by prescription. Other antibiotics, including erythromycin and bactrim are used less frequently but can work quite well. After good acne control is achieved, the oral antibiotic dosage is usually slowly lowered to the lowest level needed to keep the person acne free. See “The Acne Guide”, www.afraidtoask.com/acne/acneoral, Mar. 6, 2006.

Estrogen at a sufficient dose has been known to reduce sebum production and improve acne. The FDA has approved the triphasic oral birth-control pill (“the Pill”), which has estrogen in combination with progestin, for treatment of moderate acne. So far, there is no role for estrogen therapy in males.

Up to 90% of people with severe acne can be in complete or near complete cure within 12 to 16 weeks of isotretinoin treatment. This medication is generally reserved for those people whose acne is not responding to the above managements or if scarring is occurring. While it is an incredible medication for treating acne, it is a “big gun” and can cause a number of bothersome, serious, and even deadly side effects.

Isotretinoin's effect on acne is on several different levels. It decreases the size of sebaceous glands, and decreases the amount of bacteria that lives in other forms of treatment. Isotretinoin is currently the most effective treatment for severe pustulocystic acne that is resistant to conventional therapy. The most significant side effect is teratogenicity (birth defects), therefore isotretinoin should be used cautiously in women of childbearing age, and the drug should not be prescribed in pregnant or nursing women.

According to leading experts, acne vulgaris warranting oral isotretinoin therapy include: nodulocystic or severe inflammatory acne, acne causing severe scarring or pigmentation, acne that relapses or does not respond to conventional therapy, and acne that may affect social interactions, such as in a high profile job or adolescence.

Isotretinoin tablets are generally available in the strength of about 20 milligrams, and are usually taken twice a day with food or milk to get the best results. Typically, patients are started with about 0.5 mg/kg/day and increased to about 1 mg/kg/day, depending on the severity of the acne and tolerability. Capsules or tablets containing about 10-40 mg of isotretinoin per unit dose are standard therapy. See U.S. Pat. No. 4,545,977, col. 2, which is hereby incorporated by reference. The standard cumulative dose for isotretinoin is 120-150 mg/kg/treatment course to prevent relapse. The same dosage guidelines are applicable to repeated courses of isotretinoin. Treatment course lasts for about 4-7 months, depending on daily dose and clinical progress, but some treatments can be as short as two to four weeks with lower cumulative dosages of about 1 mg-150 mg/Kg, preferably about 60 mg/Kg. See U.S. Pat. No. 5,698,593 which is incorporated herein by reference.

Inactive ingredients for isotretinoin capsules include beeswax, butylated hydroxyanisole, edetate disodium, hydrogenated soybean oil flakes, hydrogenated vegetable oil, and soybean oil. Gelatin capsules contain glycerin and parabens (methyl and propyl), with the following dye systems: 10 mg—iron oxide (red) and titanium dioxide; 20 mg—FD&C Red No. 3, FD&C Blue No. 1, and titanium dioxide; 40 mg—FD&C Yellow No. 6, D&C Yellow No. 10, and titanium dioxide.

A number of patients have benefited by combining isotretinoin with diane (e.g., Diane 35®, antiandrogen-estrogen of cyproterone acetate-ethinyl estradiol). Since both these drugs target different mechanisms in acne pathogenesis, they act synergistically. No drug interaction between the two have been reported. See Sheth R. Poonevala V. Isotretinoin: An Indian experience. Indian J Dermatol Venereol Leprol 2001; 67:180-182.

Although diane, or Diane-35®, has many properties in common with estrogen/progestin combination oral contraceptives, it should not be prescribed solely for its contraceptive properties, according to its manufacturer. The Comprehensive Resource for Physicians, Drug and Illness Information, Diane-35® Berlex Canada; Cyproterone Acetate-Ethinyl Estradiol Acne Therapy, www.rxmed.com/b-main/62pharmaceutical/b2.1/monographs/cps-%20monographs, Mar. 6, 2006.

The currently recommended dosage schedule for the treatment of acne with isotretinoin was established sometime prior to September, 1982, when the FDA approved the use of isotretinoin by prescription. The value of this schedule was determined by treating patients with very severe cases of cystic acne who were admitted to experimental protocols testing the efficacy and toxicity of isotretinoin. Because of the demand for this drug prior to release, only the most severely affected patients were admitted into these initial clinical trials.

Currently, patients being treated have much less severe conditions for a variety of reasons. Patients with severe cystic acne who eventually relapse after treatment with isotretinoin still have much less disease than before their initial exposure to the drug, and patients are being treated in earlier stages of the disease. Dermatologists are more familiar with isotretinoin and are more comfortable in prescribing it. It is now commonplace to treat non-cystic acne with isotretinoin if the acne is chronic, antibiotic-resistant, and is associated with scarring. Most of the severe cases of acne have been treated in the past five years and are in long-term remission. Therefore, it is possible and perhaps likely that the currently used schedule is in fact excessive therapy for a portion of acne patients who have less severe, but antibiotic resistant disease.

According to U.S. Pat. No. 5,698,593, incorporated herein by reference, acne can be treated by administering to a patient having mild cystic acne or with scarring non-cystic acne a compound selected from the group consisting of isotretinoin and its derivatives, in an amount of approximately 1.5 to 3 mg/kg/day for a period of from about two to about four weeks. The treatment is then stopped, despite any persistent acne. The patients are then observed for three to four months to determine the need for additional treatment. If minimal acne remains, the short course of treatment may be repeated. If moderate or severe acne is present, then the current recommended method of 1 mg/kg/day for five months may be added.

The method of the '593 patent reduces the number of patients who must receive the full five-month course of treatment with isotretinoin or its derivatives, and thus reduces the total dose administered to patients. This, of course, reduces the duration of acute toxicity, the risk of teratogenicity, the risk of chronic radiologic toxicity, and the number of laboratory tests and physicians' office visits required to monitor the therapy. Nevertheless, even one dose of isotretinoin given to a pregnant woman can produce birth defects, so the lowered risk of teratogenicity may not be low enough.

Starting Mar. 1, 2006, to receive isotretinoin, a patient, her physician and her pharmacy must be registered in the iPLEDGE program. She may only receive up to a 30-day supply of isotretinoin at one time, and she will need a new prescription for each refill. Her prescription must be filled within 7 days of her doctor's office visit. While the iPLEDGE program is promising it can take up to an hour for a physician, patient and parent to fill out the required survey. With increased economic pressures exerted upon physicians' offices by managed care programs, the hour or so needed for each Accutane®-treated female patient is a heavy burden. Additionally, a substantial problem exists when dealing with teen-age girls or single women who may deny they are sexually active, since physicians are reluctant to disbelieve their patients.

Accordingly, there is a need to overcome the risk of birth defects associated with isotretinoin. Since isotretinoin is also being recommended for emphysema, psychotic illness and cancer, a need to avoid birth defects when being treated for these conditions exists as well.

### SUMMARY OF THE INVENTION

In a first embodiment, the present invention provides a pharmaceutical composition for the treatment of acne, comprising co-administering a therapeutically effective amount of isotretinoin and a contraceptive in a contraceptively effective daily or bi-daily dosage, wherein the contraceptive does not contain 100 wt. % progestogen (also spelled “progestagen” or “gestogen”). Progesterone is the only natural progestogen; the synthetic ones are called “progestins”.

The present invention is designed to counter the teratogenicity associated with current isotretinoin therapy. Because the pharmaceutical composition of this invention contains both isotretinoin and a contraceptive together in a daily or bi-daily dosage, the patients taking this therapy, and especially those that take an oral contraceptive for 28-56 days beforehand, are substantially prevented from conceiving. This treatment option will limit the risk of birth defects caused by patents taking isotretinoin alone, without birth control, or by physicians who prescribe isotretinoin to patients who may not be admitting that they are sexually active.

The compositions of this invention potentially provide synergistic acne therapies, since estrogen at a sufficient dose, has been known to reduce sebum reduction, and in joining together this composition with isotretinoin in a single dose, the acne can be attacked by multiple chemical and/or physiological pathways. This invention contemplates the use of contraceptives which contain estrogen, estrogen and progestagen, e.g., progesterone, progestin, or mifepristone (also known as RU486), or a combination thereof. Specifically, the estrogen can be in the form of a tri-phasic oral birth-control formulation, which has estrogen in combination with progestin.

In a further embodiment of the present invention, a method of treating acne comprising co-administering for 21 successive days, to a female of child bearing age, a combination of an oral contraceptive in a contraceptively effective daily dosage is provided. The daily dosage also includes in combination, a therapeutically effective amount of isotretinoin, followed by 4-8 days which are free of contraceptive administration, but which include continued administration of said therapeutically effective amount of isotretinoin.

In a still further embodiment of the present invention, an oral acne medication is provided having 28 separate dosage units adapted for successive daily oral administration, which comprises 21 dosage units containing in admixture with a pharmaceutically acceptable carrier, 20-50 micrograms estrogen and 0.1-3.0 mg/Kg of body weight isotretinoin, or other retinoic acid derivative equivalent, and 7 additional dosage units containing in admixture with a pharmaceutically acceptable carrier, 0.1-3.0 mg/Kg of body weight isotretinoin.

The disclosed therapies are generally but not necessarily, used by the patient in conjunction with at least one form (e.g., the Pill) and, preferably, two forms (e.g., the Pill and condom, or diaphragm and spermicide, etc.) for at least 4 weeks prior to initiation of treatment, and for at least 4 weeks after said treatment is terminated.

The oral acne medication of this invention can also be provided in 42 initial bi-daily dosage units of contraceptive and isotretinoin, followed by 14 additional bi-daily dosage units of isotretinoin. This will permit the isotretinoin to be taken as currently prescribed with meals in the morning and evening. Similarly, tri-daily dosages can be provided with proportionate dosages.

In a further embodiments of the present invention, an improved method of treating acne, including providing a blister pack comprising at least about 28 daily separate dosage units of a therapeutically effective amount of isotretinoin, or other retinoic acid derivative equivalent, and at least about 21-28 additional daily dosage units of an oral contraceptive are provided. The contraceptive is selected from the group consisting essentially of: estrogen, estrogen and protgestogen, mifepristone, or a combination thereof, for at least about 21 successive days. The oral dosage unit of isotretinoin and the oral dosage unit of the oral contraceptive are administered to a female of child-bearing age. For example, the blister pack can include 77-84 separate compartments, including at least about 28-56 separate daily dosage units of isotretinoin.

In still a further embodiment of the present invention, a blister pack, including daily dosage units arranged sequentially, includes a first phase comprising at least about 21 daily dosage units of oral contraceptive, and at least about 21 daily dosage units of isotretinoin; and a second phase of about 4-8 daily dosage units of isotretinoin. As used herein, the term “isotretinoin” also includes other retinoic acid derivative equivalents.

The isotretinoin dosages can be included in admixture with the oral contraceptive, can be delivered in a second daily or bi-daily dosage, and arranged for daily administration.

In a further embodiment of the present invention, a pharmaceutical packaging is provided which includes a first phase containing 21 separate daily dosage units of oral contraceptive which do not contain 100% progestogen, and at least 21 separate daily dosage units of isotretinoin; and a second phase comprising at least about 4-8 separate daily dosage units of isotretinoin.

In a further embodiment of the present invention, a pharmaceutical packaging is provided comprising a first phase including 21-28 separate daily dosage units of an oral contraceptive in a contraceptively effective amount, which do not contain 100% progestogen, and at least about 21-56 separate daily dosage units of isotretinoin.

In still a further embodiment of the present invention, a pharmaceutical packaging is provided including dosage units arranged sequentially and including a first phase comprising 21 separate dosage units of an oral contraceptive in a contraceptively effective amount, which does not contain 100% progestogen in admixture with isotretinoin, or other retinoic acid derivative equivalent, in a therapeutically effective amount; and a second phase comprising at least about 4-8 separate dosage units of isotretinoin.

In still a further embodiment of the present invention, a pharmaceutical package is provided comprising dosage units arranged sequentially and comprising a first phase comprising 21 separate dosage units of an oral contraceptive in a contraceptively effective amount which does not contain 100% progestogen, and at least about 21 separate dosage units of isotretinoin, or other retinoic acid derivative equivalent, in a therapeutic effective amount; and a second phase comprising at least about 4-8 separate daily dosage units of isotretinoin, or other retinoic acid derivative equivalent, in a therapeutically effective amount, without oral contraceptive administration.

It is further envisioned that the separate dosage units of oral contraceptive and isotretinoin, or other retinoic acid derivative equivalent, can be provided in separate shapes, sizes, colors, or combinations thereof.

It is further envisioned that the contraceptive and isotretinoin, or other retinoic acid derivative equivalent, ingredients can be administered transdermally, parenterally, by implant, injection, inhaler, simultaneously and/or consecutively administered, and/or in a time released fashion. Available methods of transdermal delivery include single-layer drug-in-adhesive, multi-layer drug-in-adhesive, reservoir, micro-structured transdermal, and matrix systems, for separate or simultaneous delivery of each ingredient, or both.

In addition to acne, isotretinoin has also been reported to be effective in treating psychotic illnesses such as schizophrenia, Straw, U.S. Pat. No. 4,808,630; cancer of head, neck and lung, Thomas et al, Annals of Oncology, 1999, 10, 25; Benner et al., seminars in hematology, 1994, 31, 26; and emphysema, Belloni, U.S. Pat. No. 6,794,416, which are all hereby incorporated by reference.

Since female patients being treated with isotretinoin are still at a risk for birth defects, even if such treatments are for psychotic illnesses, cancer and emphysema, the combination therapies of this invention which include co-administration with birth control contraceptives have additional utility. Moreover, patients being treated for psychotic illnesses, such as schizophrenia, can't always be depended upon to take oral contraceptives, or apply or re-apply a contraceptive patch properly or timely. For psychotic illnesses, therefore, the combined therapies of this invention are particularly suited. For lung therapy, means for delivering the 13-Cis-retinoic acid and an inhalable contraceptive directly into the lung by nebulizer, inhaler or other known delivery devices are encompassed by this invention.

### BRIEF DESCRIPTION OF THE DRAWINGS

The accompanying drawings illustrate preferred embodiments of the invention, as well as other information pertinent to the disclosure in which:

FIG. 1 is a top plan view of a blister pack containing 21 daily dosage units of isotretinoin and contraceptive in combination, and seven daily dosage units of isotretinoin;

FIG. 2 is a top plan view of a blister pack containing 28 daily dosage units of isotretinoin and 21 daily dosage units of an oral contraceptive, over a 28 day dosage cycle;

FIG. 3 is a top plan view of a blister pack containing 56 bi-daily dosage units of isotretinoin and 21 daily dosage units of an oral contraceptive over a 28 day dosage cycle;

FIG. 4 is a top plan view of a blister pack containing 56 bi-daily dosage units of isotretinoin and twenty-four daily dosage units of an oral contraceptive over a 28 day dosage cycle; and

FIG. 5 is a top plan view of a blister pack containing 56 bi-daily dosage units of isotretinoin and 28 daily dosage units of an oral contraceptive over a 28 day dosage cycle.

### DETAILED DESCRIPTION OF THE INVENTION

Oral isotretinoin (13-cis-retinoic acid or Accutane® or 13-cis vitamin A) is an isomer of all-trans retinoic acid, a metabolite of retinol (vitamin A). The absorption is enhanced when the drug is taken with food. There is no progressive accumulation of the drug in the skin during long term administration. It has a very short half-life compared to etretinate (7-37 hours). It crosses the placenta. It is rapidly eliminated by the liver and kidneys, with the main mechanism of excretion being hepatic clearance. No parent drug is identified in the urine.

Amongst both synthetic (such as etretinate, acitretin, arotinoids) and natural retinoids (such as all-trans retinoic acid and isotretinoin), only isotretinoin exerts its effect on sebum production and therefore on acne. The most likely mechanism by which isotretinoin leads to clinical improvement in acne is by reducing sebaceous gland size (up to 90%) by decreasing proliferation of basal sebocytes, suppressing sebum production and inhibiting sebocyte differentiation. Sebocyte lipid synthesis is reduced by 75% with daily doses as low as 0.1 mg/kg after four weeks. Other mechanisms include anti-inflammatory, anti-bacterial, inhibition of microbial enzyme activity and desquamation of poral occlusion.

In a first embodiment, the present invention provides a single pharmaceutical composition for the treatment of antibiotic resistant acne comprising a therapeutically effective amount of isotretinoin and a contraceptive, e.g., estrogen-containing or mifepristone-containing contraceptive, in a contraceptively effective daily or bi-daily dosage. The total number of days during which the estrogen, mifepristone, or combination of progesterone and estrogen are administered daily is preferably about 21. These are followed by 4-8 days which are preferably free of estrogen administration to approximate the natural 28-day menstrual cycle of the female. Day one of the cycle is defined as the first day of menstruation and the days are numbered sequentially thereafter until menstruation occurs again. The cycle usually lasts 28 days but it may be slightly longer or shorter. In actual practice, the pills, capsules or tablets might contain nutritional supplements such as, for example, iron supplements, folic acid, calcium, etc. Thus, in a preferred regimen, phase one would commence sometime between day 1 and day 7 of the menstrual cycle and last about 21 days.

The administration of isotretinoin and contraceptive can include separate daily or bi-daily dosage units which are adapted for successive daily or bi-daily oral ingestion. The composition consists essentially of as the first phase, about 21 daily, or 42 bi-daily, dosage units containing in admixture with a pharmaceutically acceptable carrier, a combination of mifepristone-containing or estrogen-containing contraceptive in combination with a therapeutically effective amount of isotretinoin, optionally followed by 4-8 dosage units free of contraceptive. The contraceptive and/or isotretinoin or other retinoic acid derivative daily or bi-daily dosage is preferably kept constant, or ramped up or down, in all phases. For example, patients may be started out with about 0.5 mg/Kg/day of isotretinoin for the first 7-21 days, followed by a dose of about 1.0 mg/Kg/day of isotretinoin for the next 7-21 days.

Any conventional estrogen may be employed as a suitable component in the contraceptive regimen of this invention. The particular regimen employed in a daily dosage should be equal in contraceptive activity in each phase to a daily dosage of about 20-50 μg of estrogen, such as 17α-ethinylestradiol, or estrogen in combination with progestogen, e.g., progestin, progesterone. Monophasic, biphasic and triphasic regimens including estrogen may also be used. See, for example, U.S. Pat. No. 6,214,815, incorporated herein by reference in its entirety. The preferred dosage is one equal to a daily dosage of about 25-35 μg of 17α-ethinylestradiol. Commercially available oral contraceptives useful for this purpose include Ortho-Novum 1/35 (7/7/7), Triphasil, Lo/Ovral, Tri-Levlin, to name a few.

In addition to 17α-ethinylestradiol, esters and ethers of 17α-ethinylestradiol such as, for example, 17α-ethinylestradiol 3-dimethylamino propionate, 17α-ethinylestradiol 3-cyclopentyl ether (quienestrol) and 17α-ethinylestradiol 3-methyl ether (mestranol) may also be employed as the estrogen component. Natural estrogens such as estrone, estrone sulfate, estrone sulfate piperazine salt, estradiol and estriol, and their esters, as well as the synthetic estrogens, may also be employed. A preferred estrogen is 17α-ethinylestradiol or 17α-ethinylestradiol 3-methly ether, with or without cyproterone acetate.

The estrogen, mifepristone, or estrogen/progestogen and isotretinoin components are preferably administered together orally in a pharmaceutically acceptable nontoxic carrier, but they can also be administered transdermally, separately or parenterally, by implant, injection, inhaler, simultaneously and/or consecutively administered, and/or in time released form. In general, the effective agents are processed, together with the usually additives, vehicles and/or flavor-ameliorating agents normally employed in pharmacies, in accordance with generally accepted pharmaceutical practices. For the preferred oral administration, tablets, caplets, gel-caps, soft capsules, capsules, pills, suspensions or solutions are particularly suitable; for parenteral application, oily solutions such as, for example, sesame oil or castor oil solutions which can optionally additionally contain a diluent such as, for example, benzyl benzoate or benzyl alcohol. For preferred transdermal applications, skin patches placed on the skin to deliver a time released dose of medication through the skin and into the blood stream are desirable. Types of patches include: single-layer drug-in-adhesive, multi-layer drug-in-adhesive, reservoir, microstructured transdermal systems (MTS) and matrix types. The main components to a transdermal patch are the liner, which protects the patch during storage and is generally removed prior to use; a drug, which is typically stored in solution in direct contact with the release liner; adhesive, which serves to adhere the components of the patch together along with adhering the patch to the skin; a membrane, which controls the release of the drug from the reservoir in single- and multi-layer patches; and backing, which protects the patch from the outer environment.

There are a variety of transdermal patches which have shown practical use. The single-layer drug-in-adhesive system employs an adhesive layer which also contains the drug. In this type of patch the adhesive layer not only serves to adhere the various layers together, along with the entire system to the skin, but is also responsible for releasing the drug. The adhesive layer is surrounded by a temporary liner and a backing.

In a multi-layer drug-in-adhesive patch a system similar to the single-layer patch is employed, but differs in that both adhesive layers are responsible for releasing a drug. The multi-layer system usually adds another layer of drug-in-adhesive, typically separated by a membrane (but not in all cases). This patch has a temporary liner-layer and a permanent backing. It is envisioned that the isotretinoin, or other retinoic acid derivative equivalent can be deposited within one of the adhesive layers of a multi-layer drug-in-adhesive system and the contraceptive can be deposited in the other adhesive layer, or both active ingredients can be disposed within a single adhesive layer in either a multi-layer drug-in-adhesive or single-layer drug-in-adhesive system.

Also envisioned for this invention is a reservoir system, which unlike the single-layer and multi-layer drug-in-adhesive systems, typically has a separate drug layer. The drug layer is commonly a liquid compartment containing a drug solution or suspension separated by the adhesive layer. The patch for a reservoir system is also backed by a backing layer.

Alternatively or additionally, a matrix system can be employed. This system has a drug layer of a semi-solid matrix containing a drug solution or suspension. The adhesive layer in this patch surrounds the drug layer partially overlaying it. As stated above in connection with the single-layer and multi-layer drug-in-adhesive systems, the reservoir and matrix systems can also administer the isotretinoin and contraceptive components either together in a single reservoir or matrix, or in separate reservoirs or matrices.

One patch that is already used for contraceptives is the “contraceptive patch.” This product is a transdermal patch applied to the skin that releases synthetic estrogen and progestin hormones to prevent pregnancy. Such patches are thought to have the same effectiveness as the contraceptive pill. One publicly available contraceptive patch is marketed under the brand name Ortho Evra® by Johnson and Johnson, New Brunswick, N.J. Use of this rapidly new contraceptive is increasing rapidly, likely because it combines the effectiveness of contraceptive pills with the more convenient and easy patch method of administration. Typically a woman applies the contraceptive patch onto her upper outer arm, buttocks, abdomen or thigh on either the first day of her menstrual cycle (day one) or on the first Sunday following that day, whichever she prefers. The day of application is known from that point as patch change day. Seven days later, when patch change day comes again, the woman removes the patch and applies another in its place. This process is repeated again on the next patch change day, and the following patch change day, the patch is removed and not replaced. The woman waits seven days without a patch in place normally. On the next patch change day, she can apply a new patch. If a woman chooses to begin her patch change day as day one of her menstrual cycle, the patch is able to take effect in time to prevent ovulation. In the case that a woman wishes to begin using the contraceptive patch following a first trimester abortion or miscarriage, patch application can be done immediately afterwards. This can be considered the same as a day one start above. If a woman chooses to begin with her patch change day as the first Sunday following day one, it is necessary to use one or more forms of backup contraceptive such as spermicide or condoms for the first week of patch wear.

In connection with the present invention, a woman would typically be on some form of birth control for 30 days prior to isotretinoin therapy initiation. Standard oral contraception or contraceptive patch techniques can be employed, which are administered over a 28 day cycle, involving one week (7 days) for a menstrual period. A woman would then apply a patch containing both isotretinoin or retinoic acidic derivative equivalent, and a contraceptive formulation in a contraceptively sufficient dose onto her skin on either the first day of her menstrual cycle (day 1) or on the first Sunday following that day, whichever she prefers. As with the contraceptive patch schedule mentioned above, the day of first application of the “combined patch” is known from that point as the patch change day. Seven days later, when the patch change day comes again, the woman removes the patch and applies another combined patch in its place. This process is repeated again on the next patch change day, and the following patch change day, the patch is removed and replaced with a patch containing only isotretinoin, or other retinoic acid derivative equivalent. On the next patch change day, she can apply a new patch containing both contraceptive and isotretinoin again. Preferably, once isotretinoin therapy has been started, the patch change day should not involve skipping any days of contraceptive therapy until the beginning of the menstrual cycle, during which isotretinoin therapy can continue, and alternative forms of birth control can, optionally, be used simultaneously, such as a condom, pill, sponge and/or spermicide.

Contraceptive patches are synthetic hormone contraceptives, similar in action to the contraceptive pill. The contraceptive patch product Ortho Evra® contains 0.75 mg ethinyl estradiol and 6.0 mg norelgestromin hormones in a single patch. The gradual release of hormones over the course of each week (approximately 20 micrograms/day ethinyl estradiol and 150 micrograms/day norelgestromin) act much like contraceptive pills do. Most commonly ovulation is inhibited entirely, preventing pregnancy.

In addition, other transdermal patch technology can be employed, such as microstructured transdermal systems (MTS) which use microneedle arrays for transcutaneous drug delivery. MTS has potential for providing a drug delivery solution for a wide variety of molecules, including, it is believed, estrogen/progestogen, mifepristone, estrogen and isotretinoin components, as well as vaccines, proteins and peptides. MTS provides targeted delivery to the dermal, epidermal layers of the skin. Further, MTS has the potential to enhance the efficacy of drug delivery while improving the overall delivery efficiency. Suitable transdermal technologies are disclosed in U.S. Pat. Nos. 4,751,087; 5,223,261; 5,264,219; 5,372,819; 5,380,760; 6,881,203, and 6,132,760, which are, all hereby incorporated by reference in their entirety. Transdermal products developed and manufactured by 3M are also useful and have been marketed under Minitran™, nitroglycerin transdermal delivery system, Climara® (estradiol transdermal system); Climara Pro®; (estradiol-levonorgestrel transdermal system) and Menostar®; (estradiol transdermal system). Climara® and Climara Pro® are registered trademarks of Shering A.G. and Menostar® is a registered trademark of Burlex Laboratories.

The active ingredients of this invention can be added to dispersion-type transdermal patch formulations, such as those made from acrylate copolymer adhesive, a gel-based matrix, such as lecithin gel, or a polyurethane acrylic copolymer, such as disclosed in U.S. Pat. No. 4,638,043 to Szycher et al., which is hereby incorporated by reference. Alternatively, a rate controlling membrane could be used, such as Eudraget RL-100.

In the case of the preferred oral application, the combination-type isotretinoin-contraceptives are preferably packaged in the form of a pharmaceutical kit or package in which the daily dosages are arranged for proper sequential administration. This invention also relates, therefore, to a pharmaceutical unit which contains combination-type or separated medications such as: isotretinoin-estrogen, isotretinoin-mifepristone, or isotretinoin-estrogen-progestogen, for example, in dosage units in a synchronized, fixed sequence, wherein the sequence or arrangement of the dosage units corresponds to the stages of daily administration.

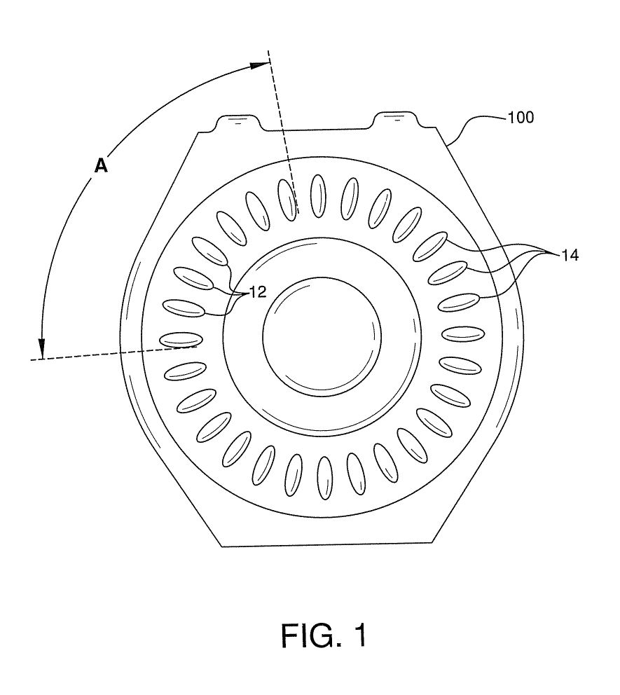

As shown in FIG. 1, the pharmaceutical unit can be, for example, in the form of a transparent package (“blister pack”) having dosage units arranged sequentially and consisting of the tablets 14 for the first phase, for example, 21 dosage units containing in admixture with a pharmaceutically acceptable carrier, 20-50 μg estrogen, more preferably 30-35 μg, and about 0.1-3.0 mg/kg of body weight isotretinoin or, most preferably about 0.5-3.0 mg/Kg, followed by the tablets 12 for the second phase, generally shown in region “A”, such as 7 additional dosage units containing 0.1-3.0 mg/kg of body weight isotretinoin or capsules or tablets of about 20-80 milligrams isotretinoin daily dosage for the average patient. A single capsule or tablet, for example, is to be taken each day over the period of the cycle. In the event a bi-daily co-administration is recommended, such as recommended widely for isotretinoin, the above dosages can be halved to 10-25 μg estrogen-containing contraceptive and 0.05-1.5 mg/kg body weight isotretinoin or other retinoic acid derivative, or alternatively about 10-40 milligrams isotretinoin bi-daily units dosage for the average patient.

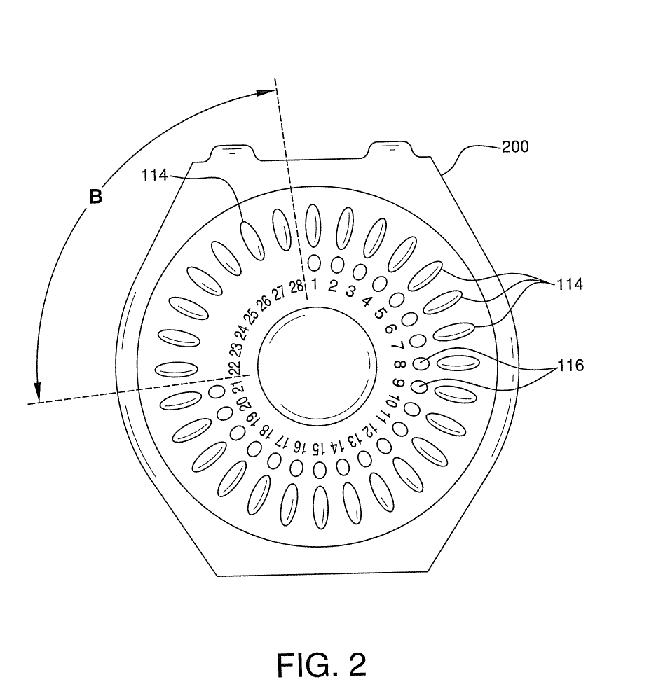

With reference to FIGS. 2-5, there are shown further pharmaceutical packages comprising dosage units arranged sequentially, which include oral contraceptives and isotretinoin or other retinoic acid derivative equivalents, for individual or separate administration, or joint or co-administration, e.g., in admixture. In the pharmaceutical package 200, shown in FIG. 2, a first phase is provided comprising 21 separate daily dosage units 116 of an oral contraceptive which does not contain 100% progestogen (generally, days numbered 1-21 for the first 21 days of administration) and at least about 21 separate daily dosage units of isotretinoin 114. The pharmaceutical package 200 also includes a second phase, generally denoted “B”, comprising at least about 4-8 separate daily dosage units 114 of isotretinoin. This second phase “B” is marked with daily increments 22-28. The pharmaceutical package 200 can also include about 4-8 placebos arranged to be taken with the 4-8 separate daily dosage units of isotretinoin of said second phase “B”.

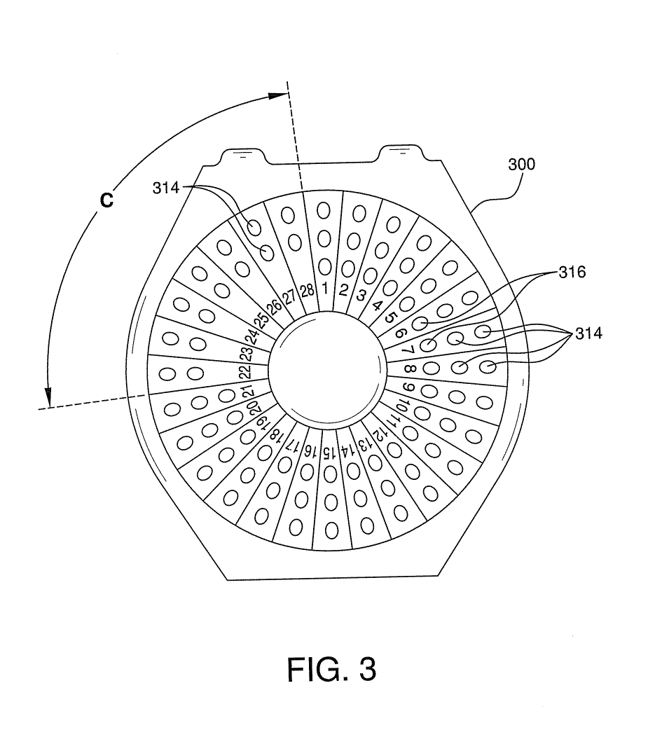

A further pharmaceutical package 300 of this invention shown in FIG. 3, a 28 day daily dosage supply is provided with a first phase lasting about 21 days, and at least about 21-56 separate daily dosage units 314 of isotretinoin. The pharmaceutical package 300 comprises at least 56 separate bi-daily dosage units of isotretinoin 314 arranged for bi-daily administration in the first phase. The pharmaceutical package 300 also has a second phase “C”, having days marked 22-28, in which bi-daily isotretinoin dosage units 314 are provided.

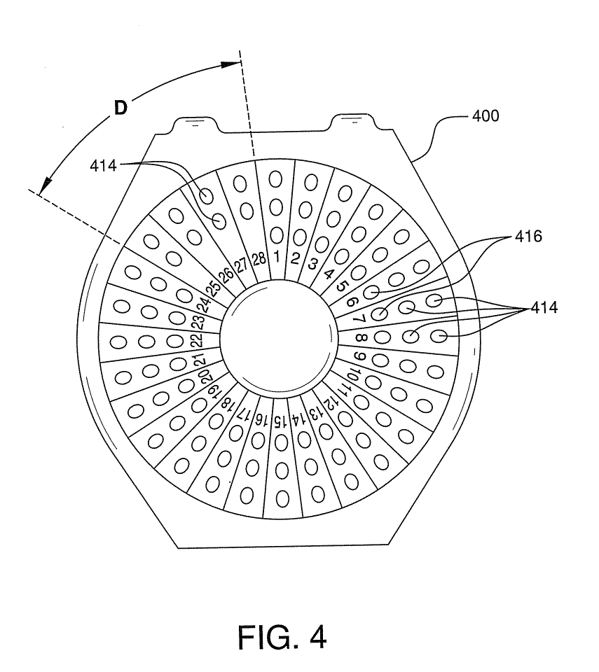

In still a further embodiment, a pharmaceutical package 400 is provided, as shown in FIG. 4. This pharmaceutical package 400 includes 28 days of pharmaceutical administration, including dosage units arranged sequentially, and comprising a first phase comprising 24 separate daily dosage units 416 of an oral contraceptive, with corresponding bi-daily dosage units 414 of isotretinoin, and a second phase marked “D”, comprising at least 4-8 separate daily dosage units of isotretinoin 414. In this case, for twenty-four days, both a bi-daily dose of isotretinoin and a daily dose of oral contraceptive are administered. In the second phase “D”, 4 days marked numerically 25-28, isotretinoin bi-daily dosages are provided. As such, the pharmaceutical package 400, provides 4 days of isotretinoin administration, without oral contraceptive administration, which provides what is known as a “shortened period”.

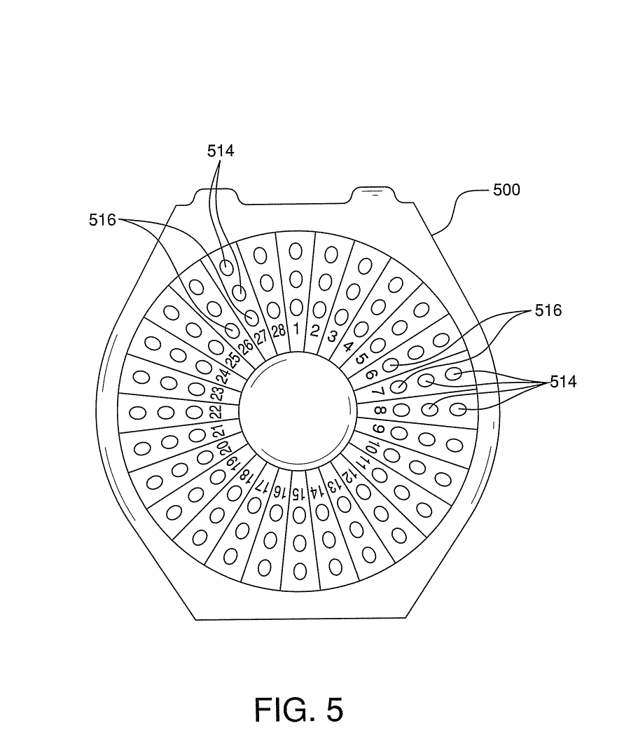

In still a further pharmaceutical package 500 of this invention, shown in FIG. 5, dosage units are arranged sequentially in a 28 day cycle, comprising 28 separate daily dosage units 516 of an oral contraceptive, and 56 separate bi-daily dosage units 514 of isotretinoin, (or alternatively 28 daily dosage units of isotretinoin).

It is understood that the separate daily dosage units of isotretinoin and the oral contraceptive can be provided in separate distinct shapes, sizes, colors and/or shades, such as round and oblong, blue and red, for example, and that further combinations of co-administered isotretinoin and oral contraceptive, along with separate daily dosage units of isotretinoin and oral contraceptive can be arranged in various cycles, including 21 days of co-administration followed by 4-8 days of isotretinoin administration, with or without a placebo, or 24-28 days of administration of both isotretinoin and oral contraceptive. The degree to which oral contraceptive is provided beyond 20 or 21 days, will generally dictate the duration of the period a woman being treated will have. From a full week of 7 days, to a shortened period of about 4 days, to none at all.

Isotretinoin therapy for acne should not be used with progesterone-only birth control hormones (examples: ‘Minipills’ like Aygestin® progestin, Micronor® progestin, Nor-QD® progestin or injectable/implantable products such as Depo-Provera® progestin or Norplant® progestin), since the Minipills may not work effectively as a contraceptive. Therefore, estrogen, e.g., monophasic and combination estrogen/progesterone, e.g., biphasic or triphasic, contraceptives are recommended.

Progestins are classified according to their structure in C19 and C21 progestagens. The C19 ones are derived from testosterone; the C21 ones from progesterone. C21 progestagens include cyproterone acetate, dydrogesterone, medroxyprogesterone acetate, chlormadinone acetate, megestrol and promegestone. Of these, dydrogesterone is structurally most similar to progesterone. C19 progestagens include norethisterone, (levo)norgestrel, lynestrenol, desogestrel, norgestimate, gestodene and tibolone.

Different progestins have different combinations of androgen (testosterone-like) and progesterone activity. If the activity of 1 mg of norethindrone is taken as the baseline, 1 mg of the other progestins have activities as shown in the following Table:

| Progestin | Progestational Activity | Androgen Activity |
|---|---|---|
| norethindrone | 1 | 1 |
| norethindrone acetate | 1.2 | 1.6 |
| desogestrel | 9 | 3.4 |
| drospirenone | 1.5 | 0 |
| ethynodiol diacetate | 1.4 | 0.6 |
| norelgestromin | 1.3 | 1.9 |
| norgestimate | 1.3 | 1.9 |
| levonorgestrel | 5.3 | 8.3 |
| dl-norgestrel | 2.6 | 4.2 |

Additionally, although newly acknowledged for its contraceptive activity, mifepristone may have utility.

In a recent study, about 3.56 million British women, approximately a third of those of reproductive age, were determined to be taking the Pill, more than 90 percent of whom are on the combined form that contains estrogen and progesterone, the two female hormones. M. Henderson, “A contraceptive pill that can beat cancer”, The Times Health News, www.timesonline.co.ulc/article/0,,2-2106558.00.html, Mar. 28, 2006. The rest were determined to be taking the mini-Pill, which contains progesterone only. The popularity of the Pill has largely recovered from the 1995 scare that prompted hundreds of thousands to give up oral contraception after “third-generation” Pills that contain different kinds of progesterone were linked to a higher risk of thrombosis.

The combined Pill protects against ovarian and endometrial tumors, but its estrogen content is thought to contribute to a slightly increased risk of breast cancer by some. While the mini-Pill does not have this drawback, it is less effective and has other side-effects, such as heavy bleeding. A new Pill using mifepristone (RU486) is designed to block the action of progesterone, which the body needs to ovulate and support a pregnancy. As it contains no estrogen, it should not promote breast cancer, and by inhibiting progesterone, it is thought that it may even reduce the risk. It is also unlikely to cause other hormonal side-effects, and has the added benefit of stopping periods, which should prevent PMS.

Mifepristone is licensed for use in abortions, though it is used at doses 100 times lower for contraception, e.g., about 2 mg. This invention preferably employs about 0.1-20 mg of mifepristone in combination with isotretinoin, preferably about 1-5 mg, for daily doses, and about 0.05-10 mg, preferably about 0.5-2.5 mg, for bi-daily doses. Mifepristone works by binding to progesterone receptors so that the body cannot respond to the hormone. If given in high doses when a woman is pregnant, it causes miscarriage, but smaller doses can prevent ovulation and conception.

From the foregoing, it can be realized that this invention provides pharmaceutical compositions for the treatment of acne, cancer, emphysema, and psychotic illnesses, comprising a co-administration of a therapeutically effective amount of isotretinoin and a contraceptive in a contraceptively effective daily or bi-daily dosage, wherein the contraceptive preferably does not contain 100 wt. % progestagen. The compositions, methods of treating acne and other diseases, and unit dosage delivery systems of this invention are designed to insure that patients taking isotretinoin are more effectively prevented from conception. The pharmaceutical compositions of this invention can be provided in blister packs containing at least 28 compartments, traditionally used for the delivery of oral contraceptives.

What is claimed is:

1. An improved method of treating acne comprising providing a blister pack comprising at least about 28 separate daily dosage units of a therapeutically effective amount of isotretinoin or other retinoic acid derivative equivalent, and at least about 21-28 additional daily dosage units of an oral contraceptive in a contraceptively effective amount selected from the group consisting of: estrogen, estrogen and progestogen, mifepristone, or a combination thereof, whereby for at least about 21 successive days, said oral dosage unit of isotretinoin or other retinoic acid derivative equivalent and said oral dosage unit of said oral contraceptive are administered to a female of child bearing age.

2. The method of claim 1 wherein said blister pack comprises at least about 77-84 separate compartments, comprising at least about 56 separate daily dosage units of isotretinoin or other retinoic acid derivative equivalent.

3. The method of claim 1 wherein said blister pack provides compartments for the administration of said isotretinoin or other retinoic acid derivative equivalent, and separate compartments for the administration of said oral contraceptive.

4. The method of claim 1 wherein said isotretinoin or other retinoic acid derivative equivalent and said oral contraceptive are provided in separate dosage units for said at least about 21 days, followed by a period of 4-8 days in which only said isotretinoin or other retinoic acid derivative equivalent dosage units are provided.

5. The method of claim 1 wherein said at least about 28 separate daily dosage units of isotretinoin comprise about 0.1-3.0 mg/kg of body weight, or about 20-80 milligrams in daily unit form.

6. The method of claim 2 wherein said at least 56 separate daily dosage units of isotretinoin or other retinoic acid derivative equivalent comprise about 0.05-1.5 mg/kg of body weight, or about 10-40 milligrams in a bi-daily unit form.

7. A pharmaceutical blister pack having dosage units arranged sequentially and comprising:
a first phase comprising at least about 21 daily dosage units of an oral contraceptive, and at least about 21 daily dosage units of isotretinoin or other retinoic acid derivative equivalent; and
a second phase of about 4-8 daily dosage units of isotretinoin or other retinoic acid derivative equivalent.

8. The pharmaceutical blister pack of claim 7 wherein said oral contraceptive does not contain 100% progestogen.

9. The pharmaceutical blister pack of claim 8, wherein each of said daily dosage of isotretinoin, or other retinoic acid derivative equivalent, comprises a bi-daily administration containing 2 units.

10. A pharmaceutical packaging comprising dosage units arranged sequentially and comprising a first phase comprising 21 separate daily dosage units of an oral contraceptive which do not contain 100% progestogen, and at least about 21 separate daily dosage units of isotretinoin, or other retinoic acid derivative equivalent, and a second phase comprising at least about 4-8 separate daily dosage units of isotretinoin, or other retinoic acid derivative equivalent.

11. The pharmaceutical package of claim 10 further comprising 4-8 placebos arranged to be taken with said 4-8 separate daily dosage units of said isotretinoin, or other retinoic acid derivative equivalent of said second phase.

12. A pharmaceutical packaging comprising dosage units arranged sequentially and comprising a first phase comprising 21-28 separate daily dosage units of an oral contraceptive in a contraceptively effective amount, which does not contain 100% progestogen, and at least about 21-56 separate daily dosage units of isotretinoin, or other retinoic acid derivative equivalent, in a therapeutically effective amount.

13. The pharmaceutical package of claim 12 comprising at least 56 separate daily dosage units of isotretinoin, or other retinoic acid derivative equivalent, and arranged for bi-daily administration.

14. The pharmaceutical package of claim 12 wherein at least 28 separate daily dosage units of said oral contraceptive are provided.

15. A pharmaceutical packaging comprising dosage units arranged sequentially and comprising a first phase comprising 21 separate dosage units of an oral contraceptive in a contraceptively effective amount which do not contain 100% progestogen, in admixture, with isotretinoin, or other retinoic acid derivative equivalent, in a therapeutically effective amount, and a second phase comprising at least about 4-8 separate dosage units of isotretinoin or other retinoic acid derivative equivalent in a therapeutically effective amount.

16. The pharmaceutical package of claim 15 wherein said oral contraceptive comprises a contraceptive selected from the group consisting of: estrogen, estrogen and progestogen, mifepristone, or a combination thereof.

17. A pharmaceutical packaging comprising dosage units arranged sequentially and comprising a first phase comprising 21 separate dosage units of an oral contraceptive in a contraceptively effective amount which does not contain 100% progestogen, and at least about 21 separate dosages units of isotretinoin, or other retinoic acid derivative equivalent, in a therapeutically effective amount, and a second phase comprising at least about 4-8 separate dosage units of isotretinoin, or other retinoic acid derivative equivalent, in a therapeutically effective amount, without oral contraceptive administration.

18. The pharmaceutical packaging of claim 17 wherein said separate dosage units of oral contraceptive and isotretinoin, or other retinoic acid derivative equivalent, are provided in daily dosages for a 21 day sequential period.

19. A method of treating acne comprising providing a transdermal patch comprising a therapeutically effective amount of isotretinoin or other retinoic acid derivative equivalent in a therapeutically effective amount and a transdermal contraceptive in a contraceptively effective amount.

20. The method of claim 19, wherein said patch is worn for about 5-9 days prior to replacement.

21. The method of claim 19, wherein said patch delivers its medication via a single-layer drug-in-adhesive, multi-layer drug-in-adhesive, reservoir, matrix type system, microstructured transdermal system or a combination thereof.

22. A medicated transdermal patch comprising,
a time released drug formulation comprising a therapeutically effective amount of isotretinoin or other retinoic acid derivative equivalent and a transdermal contraceptive in a contraceptively effective amount.

23. The transdermal patch of claim 22, wherein said patch comprises a transdermal system selected from the group consisting of: single-layer drug-in-adhesive, multi-layer drug-in-adhesive, reservoir, matrix type, microstructured transdermal system or a combination thereof.

24. The transdermal patch of claim 22, wherein said isotretinoin or other retinoic acid derivative equivalent and transdermal contraceptive are administered simultaneously.

25. The transdermal patch of claim 22, wherein said isotretinoin or other retinoic acid derivative equivalent and transdermal contraceptive are administered consecutively.

26. The transdermal patch of claim 22, wherein said isotretinoin or other retinoic acid derivative equivalent and transdermal contraceptive are administered consecutively via separate drug-in adhesive layers of a multi-layer drug-in-adhesive system for co-administration to a patient.

27. The transdermal patch of claim 22, wherein said isotretinoin or other retinoic acid derivative equivalent and transdermal contraceptive are administered consecutively via a single-layer drug-in-adhesive system for co-administration to a patient.

28. A method of treating or preventing emphysema in a patient comprising co-administering a therapeutically effective amount of isotretinoin and a contraceptively effective dosage of a contraceptive.

29. The method of claim 28 wherein said co-administration is made directly into the lung of said patient by nebulizer, inhaler, or vaporizer.

30. A method of treating or preventing psychotic illness in a patient comprising co-administering a therapeutically effective amount of isotretinoin and a contraceptively effective dosage of a contraceptive.

31. The method of claim 30 wherein said psychotic illness comprises schizophrenia.

32. A method of treating or preventing cancer in a patient comprising co-administering a therapeutically effective amount of isotretinoin and a contraceptively effective dosage of a contraceptive.

33. The method of claim 32 wherein said cancer comprises cancer of the head, neck, lung or a combination thereof.
